(19) United States
(12) Patent Application Publication
(10) Pub. No.: US 2007/0128135 A1
(43) Pub. Date: Jun. 7, 2007

(54) Use of benzoquinones in agents for oxidative treatment of hair and method and agent for permanent hair shaping

(76) Inventors: Thilo Arlt, Givisiez (CH); Pierre Alain Clement, Villars-sur-Glane (CH); Natalie Bitterli, Murten (CH)

Correspondence Address: Striker Striker & Stenby, 103 East Neck Road, Huntington, NY, 11743, US

(21) Appl. No.: 10/561,981

(22) PCT Filed: Apr. 28, 2004
(86) PCT No.: PCT/EP04/04465

§ 371 Date: Jan. 24, 2007

(30) Foreign Application Priority Data
Jun. 26, 2003 (DE) 103 28 667.5

(51) Int. Cl. A61K 8/35

(52) U.S. Cl. 424/62; 8/406; 424/70.2

(57) ABSTRACT

Use of benzoquinones and derivatives or salts thereof as the only oxidant in agents for oxidative treatment of hair, and agent containing at least one o-benzoquinone, and a method for permanent shaping of hair by use of such agents. The hair thus treated shows uniform and permanent transformation and no brightening.

The present invention relates to the use of benzoquinones in agents for oxidative hair treatment and particularly in agents for carrying out the oxidative post-treatment in permanent hair deformation, and to a method for permanent hair shaping.

In the reductive modification of keratin fibers, hair is first treated with a shaping agent that causes the opening of the disulfide bonds of hair keratin and is then brought into the desired shape. As a rule, the shaping agents used are the keratin-reducing mercapto compounds, for example the salts or esters of mercaptocarboxylic acids, or sulfites. The hair is then rinsed with water or with a suitable intermediate treatment agent. The reduced hair fibers are then subjected to an oxidative post-treatment with a fixing agent. This causes the closing of the disulfide bonds in hair keratin which is decisive for the permanent hold of the hair shaping, particularly in waving or smoothing.

Fixing agents based on hydrogen peroxide, peroxide salts (perborates, persulfates, etc) or bromates bring about the oxidation of part of the disulfide and thiol groups of hair keratin to higher oxidation states of sulfur, particularly to cysteic acid. In this manner, hair keratin is irreversibly damaged. In addition, with peroxide-containing fixing agents, the color pigments of hair (melanins) are partly destroyed which causes brightening of the hair, particularly of Asiatic hair.

The use of benzoquinone derivatives for odor improvement in hair fixing in combination with conventional oxidants (preferably bromates) is known from U.S. Pat. No. 4,560,554 (12-24-1985, to Shisedo). The use of hydrogen peroxide as the oxidant, however, causes damage to the hair as a result of overoxidation which brightens very dark hair in particular in an undesirable manner.

Hence, the object of the invention is to avoid the drawbacks arising in oxidative post-treatment, particularly in permanent hair shaping, in terms of the bleaching and cysteic acid formation without thereby deleteriously affecting the hair structure.

We have now found that this objective can be reached in outstanding manner by the use of benzoquinones in an agent for oxidative hair treatment, particularly in the post-treatment (fixation) of previously reductively treated hair for the purpose of permanent hair shaping, as defined in claim 1.

By benzoquinones are meant p-benzoquinones as well as o-benzoquinones and their water-soluble derivatives and salts.

Particularly well suited for the use according to the invention are p-benzoquinones of formula (I) and o-benzoquinones of formula (II). 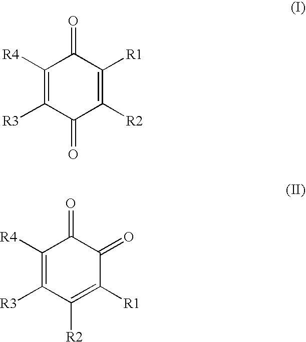
wherein
R₁, R₂, R₃, R₄ independently of each other denote —H, —C₁-C₄-alkyl, phenyl, —OH, —OR¹, NH₂,
—NHR¹, —N(R¹)₂, —N⁺(R¹)₃, —COOH, —COOR¹, —COR¹ or —SO₃H, halogen (F, CL, Br, I) or pseudohalogen (SCN, CN) wherein R¹═C₁-C₄-alkyl or phenyl.

Particularly preferred are p-benzoquinones or o-benzoquinones with at least one substituent different from hydrogen wherein R₁, R₂, R₃, R₄ denote —H, —N⁺(R¹)₃, —COOH, —COOR¹, —COR¹,

—SO₃H, halogen (F, Cl, Br, I) or pseudohalogen (SCN, CN) wherein R¹═C₁- to C₄-alkyl or phenyl.

Also suitable are p-benzoquinones or o-benzoquinones with at least one substituent different from hydrogen, wherein R₁, R₂, R₃, R₄ denote —H, -alkyl, -aryl, —OH, —OR¹, —NH₂, —NHR¹ or —NR¹₂ (R¹═C₁ to C₄-alkyl or phenyl).

Particularly preferred are water-soluble benzoquinone derivatives with one to four hydroxyl groups, such as 2-hydroxy-1,4-benzoquinone, 3-hydroxy-1,2-benzoquinone, 2,5-dihydroxy-1,4-benzoquinone, 3,4-dihydroxy-1,2-benzoquinone, tetrahydroxy-1,4-benzoquinone or tetrahydroxy-1,2-benzoquinone.

Among the benzoquinones, the o-benzoquinones and their derivatives as well as the salts thereof are particularly preferred.

In the ready-to-use agent, the benzoquinones should be used alone or in admixture with one another in an amount from 0.1 to 20 weight percent, preferably from 0.5 to 10 weight percent and particularly from 1 to 5 weight percent.

The agent for oxidative treatment of hair is preferably free of hair-damaging oxidants, particularly hydrogen peroxide, peroxide salts or bromates. Most preferably, the benzoquinones are the only oxidant present in the agent for oxidative treatment of hair. The pH of the ready-to-use agent for oxidative treatment of hair is in the range between 1.5 and 10, preferably between 2.0 and 8.0 and particularly between 2.5 and 7.5. The pH adjustment is done with at least one pH-adjusting agent such as a base, an acid or a buffer, particularly with ammonia, an ammonium or alkali metal hydroxide, an ammonium or alkali metal carbonate, an ammonium or alkali metal hydrogen carbonate, citric acid and a salt thereof (citrate buffer), phosphoric acid and a salt thereof (phosphate buffer) or ascorbic acid and salts thereof, wherein by alkali metal is preferably meant sodium or potassium, and the salts are preferably the chlorides, carbonates, sulfates or phosphates.

The ready-to-use agent for oxidative treatment of hair can be in the form of an aqueous or aqueous-alcoholic solution or an emulsion or in a water-based, thickened form, particularly a cream, gel or paste. Preferably, the benzoquinones, their derivatives or salts are used in aqueous or aqueous-alcoholic solution.

Besides the benzoquinones and the derivatives and salts thereof or a mixture thereof, the ready-to-use agent for oxidative treatment of hair can also contain additives, for example those used in cosmetic hair preparations (cf. K. Schrader, “Grundlagen und Rezepturen der Kosmetika” [Fundamentals and Formulations of Cosmetics], 2nd edition, Hüthig Verlag [publisher], Heidelberg, 1989). These include swelling agents and penetrants, for example urea, 2-pyrrolidone, 1-methyl-2-pyrrolidone and dipropylene glycol monomethyl ether, as well as acidifying agents, for example aromatic sulfonic acids, hydrochloric acid, sulfuric acid, phosphoric acid, pyrophosphoric acid or polyphosphoric acid, acid salts of strong acids, ascorbic acid, oxalic acid, malonic acid, benzoic acid, salicylic acid, citric acid and tannic acids.

Moreover, the agent for oxidative treatment of hair can contain wetting agents and emulsifiers from the group consisting of anionic, nonionic, cationic and amphoteric or zwitterionic surfactants.

Suitable surfactants are, in particular:

a) anionic surfactants, for example the alkali metal, alkaline earth metal, ammonium or alkanolamine salts of alkylsulfonates, alkylsulfates and alkyl ether sulfates, for example sodium lauryl alcohol diethylene glycol ether sulfate, sodium or triethanolamine salts of alkylsulfates with 12 to 18 carbon atoms and preferably 12 to 14 carbon atoms, the sodium or triethanolamine salts of lauryl- or tetradecyl ether sulfates and the disodium salts of the sulfosuccinate half-esters of alkanolamides, soaps and polyethercarboxylic acids;

b) nonionic surfactants, for example ethoxylated fatty alcohols with 12 to 18 carbon atoms, for example with up to 40 mole percent of ethylene oxide per mole of fatty alcohol of ethoxylated lauryl, tetradecyl, cetyl and stearyl alcohol, alone or in admixture with one another, ethoxylated lanolin alcohol ethoxylated lanolin, ethoxylated alkylphenols with 8 to 30 carbon atoms in the alkyl group and 1 to 10 ethylene oxide units in the molecule, fatty alkanolamides and ethoxylated sorbitan fatty acid esters;

c) cationic surfactants, for example dilauryldimethylammonium chloride, the chlorides or bromides of alkyldimethylbenzylammonium salts, alkyltrimethylammonium salts, for example cetyltrimethylammonium chloride or bromide, tetradecyltrimethylammonium chloride or bromide, alkyldimethylhydroxyethylammonium chlorides or bromides, dialkyldimethylammonium chlorides or bromides, alkylpyridinium salts, for example laurylpyridinium chloride or cetylpyridinium chloride, alkylamidoethyltrimethylammonium ether sulfates, compounds of a cationic character, such as amine oxides, for example alkyldimethylamine oxides or alkylolaminoethyldimethylamine oxides, and

d) amphoteric or zwitterionic surfactants, for example carboxyl derivatives of imidazole, N-alkyl- and N-alkylamidobetaines, N-alkylsulfobetaines, N-alkylaminopropionates, alkyldimethyl-carboxymethylammonium salts with 12 to 18 carbon atoms and fatty acid alkylamidobetaines, for example fatty acid amidopropyldimethylaminoacetic acid betaine.

Naturally, the agent for oxidative treatment of hair can contain all additives commonly used in such agents, for example thickeners such as kaolin, bentonite, fatty acids, higher fatty alcohols, starch, polyacrylic acid and derivatives thereof, cellulose derivatives, alginates, vaselines, or paraffin oil, moreover dyes, opacifying agents, for example polyethylene glycol esters, or alcohols, for example ethanol, propanol and isopropanol, dissolution promoters, buffering agents, perfume oils, hair-conditioning or hair-care constituents, for example lanolin derivatives, cholesterol, pantothenic acid, protein derivatives and protein hydrolyzates, betaine, provitamins and vitamins as well as plant extracts.

To prepare the ready-to-use cosmetic agent for oxidative treatment of hair, the constituents of the cosmetic preparation are used in amounts commonly employed for this purpose. For example, wetting agents and emulsifiers are used at a concentration of 0.2 to 30 weight percent, alcohols at a concentration of 1 to 80 wt. %, hair-conditioning and hair-care constituents at a concentration of 0.1 to 10 wt. % and thickeners at a concentration of 0.1 to 25 wt. %, based on the ready-to-use fixing agent.

The agent used for oxidative treatment of hair is preferably a long-term permanent hair deformation fixing agent (in the following referred to as “fixative”) or an oxidation hair colorant, the former being particularly preferred.

The ready-to-use agent for oxidative treatment of hair is employed in a temperature range from 10 to 60° C., preferably from 20 to 55° C. and particularly from 20 to 40° C. Treatment times range from 1 to 45 minutes, preferably from 2 to 25 minutes and particularly from 3 to 15 minutes.

Most preferably, the agent used for oxidative treatment of hair has a pH in the range from 1.5 to 10, particularly from 2.0 to 8.0 and especially from 2.5 to 7.5. Said agent contains (A) 0.5 to 10.0 wt. % of at least one benzoquinone, a derivative or salt thereof and B) 0.01 to 10 wt. % of at least one pH-adjusting agent, for example ammonia, an ammonium or alkali metal hydroxide, an ammonium or alkali metal carbonate, an ammonium or alkali metal hydrogen carbonate, citric acid and a salt thereof (citric buffer), phosphoric acid and a salt thereof (phosphate buffer), or ascorbic acid and the salts thereof, the preferred alkyl metals being sodium or potassium and the preferred salts being the chlorides, carbonates, sulfates or phosphates.

Another object of the present invention is an agent for oxidative treatment of hair, characterized in that it contains at least one o-benzoquinone selected from among o-benzoquinone and the derivatives or salts thereof. The o-benzoquinone preferably has the general formula (II). Most preferably, the o-benzoquinone derivative is selected from among 3-hydroxy-1,2-benzoquinone, 3,4-dihydroxy-1,2-benzoquinone and tetrahydroxy-1,2-benzoquinone.

Most preferably, the agent used for oxidative treatment of hair has a pH in the range from 1.5 to 10, particularly from 2.0 to 8.0 and especially from 2.5 to 7.5. Said agent contains (A) 0.5 to 10.0 wt. % of o-benzoquinone selected from among o-benzoquinone and derivatives and salts thereof and B) 0.01 to 10 wt. % of at least one pH-adjusting agent, particularly ammonia, an ammonium or alkali metal hydroxide, an ammonium or alkali metal carbonate, an ammonium or alkali metal hydrogen carbonate, citric acid and a salt thereof (citric buffer), phosphoric acid and a salt thereof (phosphate buffer), or ascorbic acid and the salts thereof, the preferred alkali metals being sodium or potassium and the preferred salts being the chlorides, carbonates, sulfates or phosphates.

Otherwise, the agent according to the invention can contain the same common additives in the same amounts as indicated in the foregoing for the agent for oxidative treatment of hair used according to the invention.

Another object of the present invention is a method for permanent hair shaping whereby the hair before and/or after it was brought into the desired shape is treated with a keratin-reducing deformation agent, optionally rinsed and then oxidatively post-treated with a fixative, optionally rinsed and finally shaped into a hairstyle and then dried, characterized in that the aforedescribed agent for post-oxidative treatment of hair is used as the fixative. The rinsing is preferably done with water.

In a particular embodiment of the method of the invention, the hair is first treated with the keratin-reducing shaping agent. After the treatment period, the shaping agent is rinsed out after which the hair is treated with the aforedescribed agent for oxidative treatment of hair based on benzoquinones or derivatives or salts thereof as oxidant (prefixed) and then post-treated (post-fixed) with a fixative based on hydrogen peroxide or a bromate.

Particularly advantageously, the fixative for the post-fixing has a lower concentration of oxidant than is usual for such agents. For example, the concentration of hydrogen peroxide is only 0.1 to 1 wt. % and that of bromate only 1 to 5 wt. %.

According to the method of the invention, the hair is washed, rubbed with a towel, optionally premoistened with part of the keratin-reducing shaping agent, divided into individual strands and wound onto rollers [“perm rods”]. The diameter of the rollers is either about 5 to 13 millimeters or about 15 to 35 millimeters, depending on whether permanent waving or defrizzing is desired. Subsequently, an amount of shaping agent sufficient for permanent hair shaping is applied onto the rolled-on hair. The total amount of shaping agent required for permanent hair shaping is, in general, about 80 to 120 grams.

The shaping agents commonly used by the method of the invention contain conventional keratin-reducing compounds, for example certain mercapto compounds, particularly thioglycolic acid or salts thereof, thiolactic acid or salts thereof, cysteine, cysteamine and the salts or esters of mercaptocarboxylic acids. These shaping agents contain the keratin-reducing compounds in an amount common for such agents, for example the ammonium salts of thioglycolic acid or thiolactic acid in an amount of about 2 to 12 weight percent. The pH of the shaping agent is, in general, about 7 to 11, the pH adjustment preferably being done with ammonia, mono-ethanolamine, ammonium carbonate or ammonium hydrogen carbonate. For shaping agents adjusted to an acidic pH (for example pH=6.5 to 6.9), esters of mercaptocarboxylic acids, for example monothioglycolic acid ethylene glycol esters or glycerol esters, or sulfites at a concentration of about 2 to 25 weight percent are preferably used.

Moreover, the shaping agent can contain all additives commonly used in such agents, for example swelling agents and penetrants, thickeners, wetting agents and emulsifiers, alcohols, dissolution promoters, stabilizers, dyes, perfume oils as well as hair-conditioning or hair-care constituents. The aforesaid additives are used in amounts commonly employed for such purposes, for example the wetting agents and emulsifiers at a concentration of about 0.2 to 30 weight percent, whereas the thickeners can be contained in the shaping agent in an amount of about 0.1 to 25 weight percent.

The shaping agent used by the method of the invention can be in the form of an aqueous solution or emulsion or in a water-based thickened form, particularly a cream, gel or paste or in the form of an aerosol foam.

After a treatment time sufficient for permanent shaping which depending on the hair structure pH, shaping efficiency of the shaping agent and the use temperature is from about 5 to 45 minutes (5 to 30 minutes if heat is used; 20 to 45 minutes without the use of heat), the hair is rinsed with water and then oxidatively post-treated with about 50 to 350 grams and preferably 80 to 200 grams of the aforedescribed ready-to-use fixing agent.

After a period of treatment with the fixing agent of 1 to 45 minutes, preferably 3 to 25 minutes and particularly 5 to 15 minutes, the rollers are removed, and the unrolled hair, only if necessary, is once again oxidatively post-treated with the fixing agent. The hair is then rinsed, preferably with water, shaped into a hairstyle and dried.

The hair treated in this manner shows a uniform and holding transformation. Unlike for hair fixed with peroxide and particularly for Japanese hair which shows a clearly noticeable color shift in the direction of red and yellow (degree of brightening), the brightening values for the fixation according to the invention lie in the range of untreated hair strands. Moreover, the cysteic acid content of hair thus treated is clearly below that of hair treated with the fixing agent based on hydrogen peroxide or a bromate.

### EXAMPLES

The following examples are intended to give an accurate description of the method of the invention.

In all these examples, the hair was reductively pretreated in the following manner:

Three to four untreated and, hence, not previously damaged counted hair strands (consisting either of 100 medium-brown European hairs or of 60 black, Asiatic hairs, each having a length of exactly 16.5 centimeters) were rolled wet onto standardized spiral rollers (inner diameter: 3 millimeters) and after conditioning in a desiccator (temperature: 20° C.; rel. humidity: 99%) treated with a commercial waving lotion (thioglycolic acid content: 10 weight percent; pH=8.2). The amount of applied waving lotion was calculated on the basis of a 1:1.2 ratio (1 g of hair: 1.2 mL of waving lotion. The amount of 1.2 mL per about 1 g of hair corresponds to an amount of 50 mL of waving lotion per lady's head with an average weight of about 30 g of hair per head. The treatment period selected was 15 minutes, and the treatment temperature of the reducing agent was 50 degrees centigrade.

### Example 1

At the end of the treatment period, excess waving lotion was rinsed out with water, and the following fixing solution was applied:

| | |
|---|---|
| 1.00 g | of p-benzoquinone |
| 0.04 g | of phosphoric acid |
| to 100.00 g | water |

The pH of the agent was 2.5.

The hair was fixed for 10 minutes at 22 degrees centigrade and then rinsed with water.

The rollers were then removed, and the hair was rinsed with lukewarm water. The hair was finally set to a hairstyle and then dried.

The hair treated in this manner was in a good general state, was not brightened and showed an increased luster.

### Example 2

As in Example 1, at the end of the treatment period, excess waving lotion was rinsed out with water after which the following fixing solution was applied:

| | |
|---|---|
| 1.00 g | of o-benzoquinone |
| 0.02 g | of ammonia, 25% aqueous lotion |
| to 100.00 g | water |

The pH of the agent was 6.

The hair was fixed for 10 minutes at 22° C. and then rinsed with water. The rollers were then removed, and the hair was rinsed with lukewarm water. The hair was finally set to a hairstyle and then dried.

The hair thus treated showed a good transformation and hold, was not brightened and had an increased luster.

### Example 3

As in Example 1, at the end of the treatment period, excess waving lotion was rinsed out with water after which the following fixing solution was applied:

| | |
|---|---|
| 1.00 g | of 2,5-hydroxy-1,4-benzoquinone |
| 2.14 g | of ammonia, 25% aqueous lotion |
| to 100.00 g | water |

The pH of this agent was 6.

The hair was fixed for 10 minutes at 22° C. and then rinsed with water. The rollers were then removed, and the hair was rinsed with lukewarm water. The hair was finally set to a hairstyle and then dried.

The hair treated in this manner shows a good transformation and hold, was not brightened and showed increased luster.

### Example 4

As in Example 1, at the end of the treatment period, excess waving lotion was rinsed out with water after which the following fixing solution was applied:

| | |
|---|---|
| 1.00 g | of tetrahydroxy-1,4-benzoquinone |
| 0.38 g | of ammonia, 25% aqueous solution |
| to 100.00 g | water |

The pH of this agent was 6.

The hair was fixed for 10 minutes at 22° C. and then rinsed with water. The rollers were then removed, and the hair was rinsed with lukewarm water. The hair was finally set to a hairstyle and then dried.

The hair treated in this manner showed a good transformation and hold, was not brightened and showed increased luster.

1. Use of benzoquinones as the only oxidant in agents for oxidative treatment of hair.

2. Use as defined in claim 1, characterized in that the benzoquinone used is p-benzoquinone, o-benzoquinone or a water-soluble derivative or salt thereof.

3. Use as defined in claim 2, characterized in that the water-soluble benzoquinone derivative is dihydroxybenzoquinone or tetrahydroxybenzoquinone.

4. Use as defined in claim 3, characterized in that the benzoquinone derivative used is 2-hydroxy-1,4-benzoquinone, 3-hydroxy-1,2-benzoquinone, 2,5-dihydroxy-1,4-benzoquinone, 3,4-dihydroxy-1,2-benzoquinone, tetrahydroxy-1,4-benzoquinone or tetrahydroxy-1,2-benzoquinone.

5. Use as defined in one of claims 1 to 4, characterized in that the benzoquinone, the salt or the derivative thereof is used in an amount from 1 to 5 weight percent.

6. Use as defined in one of claims 1 to 5, characterized in that the benzoquinone, the derivative or the salt thereof is used at a pH of 2.5 to 7.5.

7. Use as defined in one of claims 1 to 6, characterized in that the agent for oxidative treatment of hair is a permanent hair-shaping fixing agent.

8. Agent for oxidative treatment of hair, characterized in that it contains at least one o-benzoquinone, selected from among o-benzoquinone and derivatives or salts thereof.

9. Agent for oxidative treatment of hair as defined in claim 8, characterized in that it has a pH in the range from 1.5 to 10 and contains:
(A) 0.5 to 10.0 wt. % of at least one o-benzoquinone, a derivative thereof or a salt thereof, and
(B) 0.01 to 10 wt. % of at least one pH-adjusting agent.

10. Agent as defined in claim 9, characterized in that the pH-adjusting agent is selected from among ammonia, an ammonium or alkali metal hydroxide, an ammonium or alkali metal carbonate, an ammonium or alkali metal hydrogen carbonate, citric acid and the salts thereof, phosphoric acid and the salts thereof or ascorbic acid and the salts thereof, wherein alkali metal denotes sodium or potassium and the salts are chlorides, carbonates, sulfates or phosphates.

11. Method for permanent hair shaping whereby the hair, before and/or after it has been brought into the desired shape, is treated with a keratin-reducing shaping agent, after which it is rinsed, then oxidatively post-treated with a fixing agent and again rinsed, characterized in that the fixing agent used is an agent as defined in claim 8.
